(19) United States
(12) Patent Application Publication
(10) Pub. No.: US 2024/0299637 A1
(43) Pub. Date: Sep. 12, 2024

(54) CONTACTLESS PROXIMITY CONTROLS FOR A MEDICAL DEVICE

(71) Applicants: Fresenius Medical Care Holdings, Inc., Waltham, MA (US); Fresenius Medical Care Deutschland GmbH, Bad Homburg (DE)

(72) Inventors: Christian Schlaeper, Wehrheim (DE); David Yuds, Hudson, NH (US); Martin Joseph Crnkovich, Walnut Creek, CA (US)

(21) Appl. No.: 18/182,083

(22) Filed: Mar. 10, 2023

(51) Int. Cl. A61M 1/16

(52) U.S. Cl. CPC A61M 1/1601; A61M 2205/18; A61M 2205/3584

(57) ABSTRACT

Systems and methods are described for a dialysis machine (such as a hemodialysis machine) with proximity and motion sensors used to control simple functions of the dialysis machine through contactless interfacing that would otherwise normally be handled with glove changes. The sensors may be used to perform the same functions as using the touch screen display or the control panel of the dialysis machine. In an implementation, one or more of the sensors may be a proximity sensor that receives short-range wireless signals from a credential device of an operator, and one or more of the sensors may be a motion sensor enabling the operator to move a hand or foot near the sensor to enable contactless interfacing with the dialysis machine.

### TECHNICAL FIELD

This application relates generally to contactless proximity controls for a medical device, particularly a dialysis machine.

### BACKGROUND

Medical devices, such as dialysis machines, are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. Before the blood is returned to the body, air bubbles are removed from the blood to inhibit embolisms. The process of removing air is typically accomplished through use of a venous drip chamber, which is located downstream of the blood outlet of a dialyzer and upstream of the venous blood return of the patient. During peritoneal dialysis, the patient's peritoneal cavity is periodically infused with dialysate (or dialysis solution). The membranous lining of the patient's peritoneum acts as a natural semi-permeable membrane that allows diffusion and osmosis exchanges to take place between the solution and the blood stream. Automated peritoneal dialysis machines, also called PD cyclers, are designed to control the entire peritoneal dialysis process so that it can be performed at home, usually overnight, without clinical staff in attendance. Both HD and PD machines may include displays with touch screens or other user interfaces that display information of a dialysis treatment and/or enable an operator or patient to interact with the machine.

HD machines in dialysis clinics (in-center) are usually grouped into pods of four or more machines to allow one clinician to monitor several treatments at one time. Within that station is a Medical Information System (MIS), hand sanitizer, and boxes of sterile gloves. This grouping allows the monitoring clinician to quickly respond to alarms and other patient needs to deliver the best possible care with minimal resources for maximum efficiency. Preventing the spread of infectious diseases is undertaken by using the sterile gloves and hand sanitizer.

When an alarm occurs on one of the hemodialysis machines, the clinician responds by muting the alarm, reviewing and troubleshooting its cause, and resetting the system with a Reset button, which may be a touchscreen button or physical key. If part of the troubleshooting requires handling the patient, the clinician must change gloves. If the clinician needs to touch the machine again, the clinician must change gloves. If the machine mute times out and the alarm sounds again, the clinician must change gloves yet again after touching the machine. When the clinician is finished with the event, hand sanitizer and new, sterile gloves are used to prepare for the next event. This routine is multiplied by the number of HD machines in the pod and the frequency of the alarms. There can be more than one machine alarming at any time. The necessary changing of gloves and application of copious amounts of hand sanitizer slows down the response time, causes wear and tear on the clinician's hands, and creates a lot of trash. Touch free machine operation using remote control is possible but can lead to problems with the remote control getting lost, dirty, or running out of batteries.

Accordingly, it would be desirable to provide a system that addresses the above-noted concerns and other issues.

### SUMMARY

According to the system described herein, a medical system for conducting a medical treatment includes a medical device for performing the medical treatment and having a display and at least one motion sensor coupled to the medical device. The at least one motion sensor includes a contactless interface that is configured to enable contactless interaction by an operator with the medical device. The display of the medical device identifies an action at the medical device that is enabled by interaction with the contactless interface of the at least one motion sensor.

According further to the system described herein, a method for contactless interfacing with a medical device configured to perform a medical treatment includes displaying an alarm concerning the medical treatment on a display of the medical device and activating at least one motion sensor that is coupled to the medical device. The at least one motion sensor includes a contactless interface configured to enable contactless interaction by an operator to interact with the medical device to respond to the alarm, wherein an action at the medical device caused by the operator interacting with the contactless interface of the at least one motion sensor is identified on the display of the medical device.

In various implementations, the system and/or method described herein may further include one or more of the following features. The contactless interface of the at least one motion sensor may include an ultrasonic sensor. The at least one motion sensor may be disposed at a base of the dialysis machine and configured to respond to motion of a foot of the operator. The at least one motion sensor may include at least two motion sensors coupled at different locations on the medical device, and wherein a first motion sensor of the at least two motion sensors corresponds to, and is positioned remotely from, a first button displayed on the display of the medical device, and wherein a second motion sensor of the at least two motion sensors corresponds to, and is positioned remotely from, a second button displayed on the display of the medical device. The action may include one or more of muting or resetting an alarm at the medical device and/or the action may include modifying a parameter of the medical device to respond to an alarm at the medical device. A proximity sensor may be coupled to the medical device, wherein the proximity sensor is configured to receive short-range wireless signals that may include WiFi, Bluetooth or RFID signals. A credential device may be included that provides a wireless signal containing credential information of the operator to the proximity sensor. The credential information of the operator may be verified via the medical device before the at least one motion sensor is activated to enable the operator to interact with the contactless interface of the at least one motion sensor. The at least one motion sensor may be configured to recognize a specific gesture by the operator before enabling further contactless interaction with the by the operator with the medical device.

### BRIEF DESCRIPTION OF THE DRAWINGS

Embodiments and features of the system described herein are explained with reference to the several figures of the drawings, which are briefly described as follows.

FIG. 1 is a schematic illustration of a dialysis machine configured with proximity and motion sensors in accordance with an implementation of the system described herein.

FIG. 2 is a block diagram of an example computer system illustrated in connection with the dialysis machine.

FIG. 3 is a schematic illustration showing features and functions of the dialysis machine having proximity controls according to the system described herein in connection with occurrence of an alarm at the dialysis machine.

FIG. 4 is a schematic illustration showing an example operation of the system described herein according to an implementation thereof involving contactless interaction by an operator.

FIG. 5 is a flow diagram showing an example workflow for actions and/or processing performed by, and contactless engagement between, the dialysis machine and an operator concerning an alarm.

### DETAILED DESCRIPTION

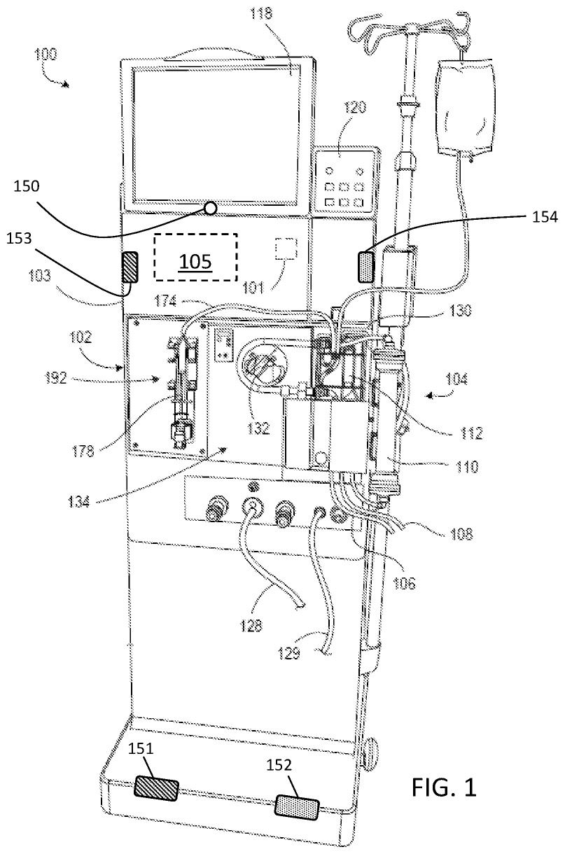

FIG. 1 is a schematic illustration of a dialysis machine configured with proximity and motion sensors in accordance with an implementation of the system described herein. A hemodialysis system 100 is shown that includes a dialysis machine 102 (e.g. a hemodialysis machine) connected to a disposable blood component set 104 that partially forms a blood circuit. During hemodialysis treatment, an operator connects arterial and venous patient lines 106, 108 of the blood component set 104 to a patient. The blood component set 104 may include an air management device 112 that may include, for example, a venous drip chamber.

The blood component set 104 is secured to a module 130 attached to the front of the dialysis machine 102. The module 130 includes the blood pump 132 capable of circulating blood through the blood circuit. The module 130 also includes various other instruments capable of monitoring the blood flowing through the blood circuit. The module 130 includes a door that when closed, as shown in FIG. 1, cooperates with the front face of the module 130 to form a compartment that is sized and shaped to receive the blood component set 104.

The blood pump 132 is part of a blood pump module 134. The blood pump module 134 includes a display window, a start/stop key, an up key, a down key, a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in a drip chamber.

The dialysis machine 102 further includes a dialysate circuit formed by the dialyzer 110, various other dialysate components, and dialysate lines connected to the dialysis machine 102. Many of these dialysate components and dialysate lines are inside the housing 103 of the dialysis machine 102 and are thus not visible in FIG. 1. During treatment, while the blood pump 132 circulates blood through the blood circuit, dialysate pumps (not shown) circulate dialysate through the dialysate circuit.

A drain line 128 and an ultrafiltration line 129 extend from the dialysis machine 102. The drain line 128 and the ultrafiltration line 129 are fluidly connected to the various dialysate components and dialysate lines inside the housing 103 of the dialysis machine 102 that form part of the dialysate circuit. During hemodialysis, the dialysate supply line carries fresh dialysate to the portion of the dialysate circuit located inside the dialysis machine 102. As noted above, the fresh dialysate is circulated through various dialysate lines and dialysate components, including the dialyzer 110, that form the dialysate circuit. As the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate (described below) and excess fluid drawn from the patient is carried to the drain via the ultrafiltration line 129.

The dialyzer 110 serves as a filter for the patient's blood. The dialysate passes through the dialyzer 110 along with the blood, as described above. A semi-permeable structure (e.g., a semi-permeable membrane and/or semi-permeable microtubes) within the dialyzer 110 separates blood and dialysate passing through the dialyzer 110. This arrangement allows the dialysate to collect toxins from the patient's blood. The filtered blood exiting the dialyzer 110 is returned to the patient. The dialysate exiting the dialyzer 110 includes toxins removed from the blood and is commonly referred to as “spent dialysate.” The spent dialysate is routed from the dialyzer 110 to a drain.

A drug pump 192 also extends from the front of the dialysis machine 102. The drug pump 192 is a syringe pump that includes a clamping mechanism configured to retain a syringe 178 of the blood component set 104. The drug pump 192 also includes a stepper motor configured to move the plunger of the syringe 178 along the axis of the syringe 178. A shaft of the stepper motor is secured to the plunger in a manner such that when the stepper motor is operated in a first direction, the shaft forces the plunger into the syringe, and when operated in a second direction, the shaft pulls the plunger out of the syringe 178. The drug pump 192 can thus be used to inject a liquid drug (e.g., heparin) from the syringe 178 into the blood circuit via a drug delivery line 174 during use, or to draw liquid from the blood circuit into the syringe 178 via the drug delivery line 174 during use.

The dialysis machine 102 includes a user interface with input devices such as a touch screen or display 118 and a control panel 120. The touch screen display 118 and the control panel 120 allow the operator to input various different treatment parameters to the dialysis machine 102 and to otherwise control the dialysis machine 102. The touch screen display 118 displays information to the operator of the hemodialysis system 100.

The dialysis machine 102 includes a control unit 101 (e.g., a processor) configured to receive signals from and transmit signals to the touch screen display 118 and the control panel 120. The control unit 101 can control the operating parameters of the dialysis machine 102, for example, based at least in part on the signals received by the touch screen display 118 and the control panel 120. The dialysis machine 102 may also include a communication unit 105 that may be provided for wireless communication with a remote computer and/or cloud infrastructure using a network and may be communicatively coupled with the control unit 101. In an implementation, as further described in detail elsewhere herein, the dialysis machine 102 includes a proximity sensor 150 having wireless-signal transceiver capability and motion sensors 151, 152, 153, 154.

Although the system is described herein principally in connection with a particular configuration of hemodialysis machine, the system described herein may be used and implemented in connection with other configurations or types of hemodialysis machines, including, for example, one or more machines from the 2008 and 5008 series of dialysis machines manufactured by Fresenius Medical Care, as well as other medical devices, including peritoneal dialysis machines, infusion pumps, heartrate monitors, etc. that would benefit from the communication and control features described herein.

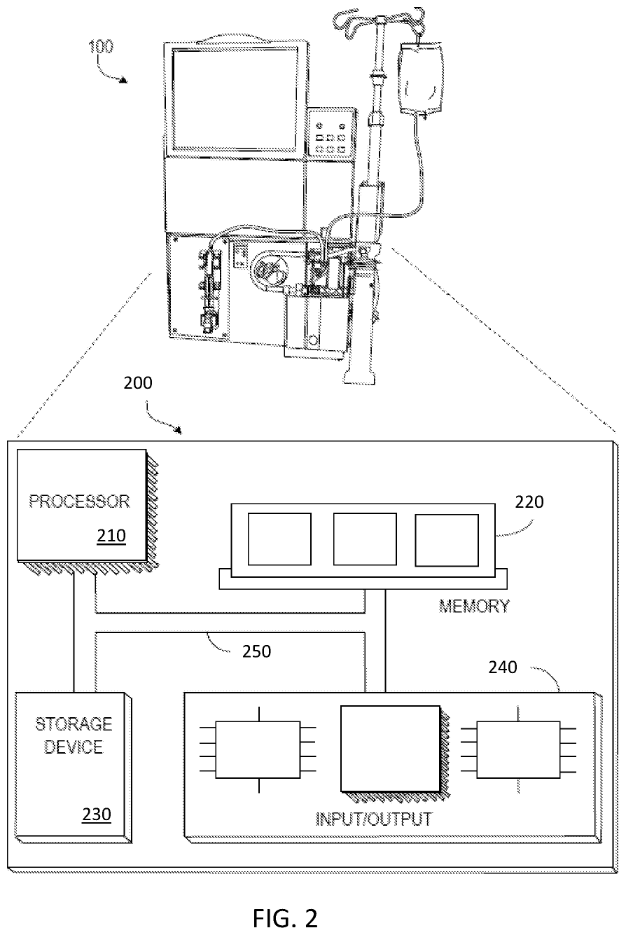

FIG. 2 is a block diagram of an example computer system 200 illustrated in connection with the dialysis machine 102. For example, the control unit 101 discussed above could be an example of the system 200 described here. The system 200 includes a processor 210, a memory 220, a storage device 230, and an input/output device 240. Each of the components 210, 220, 230, and 240 can be interconnected, for example, using a system bus 250. The processor 210 is capable of processing instructions for execution within the system 200. The processor 210 can be a single- threaded processor, a multi-threaded processor, and/or other computer. The processor 210 is capable of processing instructions stored in the memory 220 or on the storage device 230. The memory 220 stores information within the system 200. In some implementations, the memory 220 is a computer-readable medium. The memory 220 can, for example, be a volatile memory unit or a non-volatile memory unit.

The storage device 230 is capable of providing mass storage for the system 200. In some implementations, the storage device 230 is a non-transitory computer-readable medium. The storage device 230 can include, for example, a hard disk device, an optical disk device, a solid-state drive, a flash drive, magnetic tape, or some other large capacity storage device. The storage device 230 may alternatively be a cloud storage device, e.g., a logical storage device including multiple physical storage devices distributed on a network and accessed using a network. In some implementations, the information stored on the memory 220 can also or instead be stored on the storage device 230.

The input/output device 240 provides input/output operations for the system 200. In some implementations, the input/output device 240 includes one or more of network interface devices (e.g., an Ethernet card), a serial communication device (e.g., an RS-232 10 port), and/or a wireless interface device (e.g., a Bluetooth, WIFI or other short-range wireless communication device, a wireless modem). In some implementations, the input/output device 240 includes driver devices configured to receive input data and send output data to other input/output devices, e.g., a keyboard, a printer, and display devices (such as the touch screen display 118). In some implementations, mobile computing devices, mobile communication devices, and other devices are used. The input/output device 240 may further include the communication component 105 that is discussed in more detail elsewhere herein. In some implementations, the system 200 is a microcontroller. A microcontroller is a device that contains multiple elements of a computer system in a single electronics package. For example, the single electronics package could contain the processor 210, the memory 220, the storage device 230, and input/output devices 240.

According to the system described herein, sensors provided at a dialysis machine may be used to control simple functions of the dialysis machine that would otherwise normally be handled with quick glove changes, including in connection with actions of an operator to respond to an alarm. The sensors may be used to perform the same functions as using the touch screen display or the control panel. In one or more implementations, the sensors may be ultrasonic so the operator may place a hand or foot near the sensor to trigger the dialysis machine response in a contactless manner. Other appropriate motion sensing technologies may also be used in connection with the system described, such as passive infrared technology.

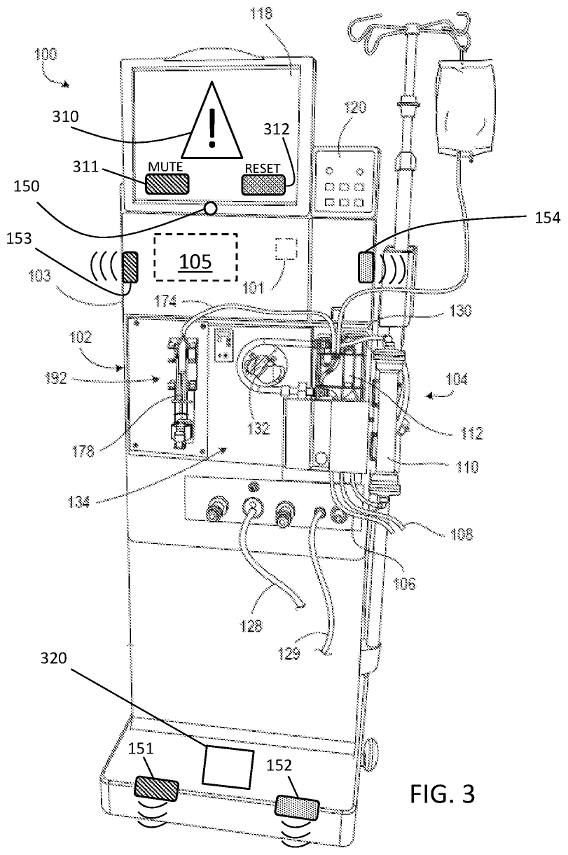

FIG. 3 is a schematic illustration showing features and functions of the dialysis machine 102 having proximity controls according to the system described herein in connection with occurrence of an alarm at the dialysis machine 102. For example, the alarm, such as a venous drip chamber low alarm, may be triggered at the dialysis machine 102, and shown as a visual alarm 310 on the display 118 of the dialysis machine 102 (other indications of the alarm event, such as audible alarm alerts may also occur). The display 118 may further show buttons for addressing the alarm on the display 118, such a mute button 311 or a reset button 312 which may be activated by a nurse or technician who is responding to the alarm. In normal course, responding to the alarm may include pushing one or more of the buttons on the touchscreen display 118 or using keys on the control panel 120. In an implementation of the system described herein, the sensors 151 and 152 may comprise motion sensors provided at the base of the dialysis machine 102 that may determine a motion or gesture of a foot in proximity to the sensors and used to control simple functions of the dialysis machine 102 that would otherwise need to be handled with glove changes. In an implementation the sensors 151, 152 may provide for activating functions of a mute button or a reset button in a manner that is accessible at the base of the dialysis machine 102, in addition to accessibility via buttons 311, 312 on the display 118. The mute and reset operations may perform the same functions as the buttons 311 and 312 on the display 118. In an implementation, the sensors 151, 152 may be ultrasonic enabling the operator to bring her foot at or near the sensor 151 or the sensor 152 to trigger the corresponding dialysis machine response to the alarm, e.g. mute or reset. Additionally and/or alternatively, motion sensors 153, 154 may be disposed on the sides of the dialysis machine 102 in a position suitable to detect hand or gesture motion by an operator to trigger the corresponding dialysis machine response to the alarm, e.g. mute or reset.

An additional layer of security may be provided by the proximity sensor 150 disposed on the front of the dialysis machine 102 that may be used as an identification system used to establish that a person, and not an object, has triggered any of the motion sensors 151, 152, 153, 154, and/or as a verification system used to control or verify authorization of the operator responding to the alarm. For example, in various implementations, the proximity sensor 150 may include NFC, Bluetooth and/or RFID transceivers that may be used to confirm the identity and/or verify the credentials of the operator before allowing activation of the motion sensors 151, 152, 153, 154 for interacting with the dialysis machine 102. For example, the NFC, Bluetooth and/or RFID transceivers may receive a signal from a chip in the operator's smartcard, work badge and/or smartphone and process the signal to identify and/or verify the operator's credentials. This system has the added benefit of keeping a log or other record of who responds to which alarms. The log may be transmitted to a remote computer or system and/or uploaded to the cloud, such as by using the communication unit 105 of the dialysis machine 102, along with other records of alarm events or treatment interventions. Alternatively and/or additionally the proximity sensor 150 may include facial recognition capabilities, such as a camera, that may be used, for example in connection with the processor 101, to identify the operator of the dialysis machine 102 and/or authorize the operator to interact with the dialysis machine 102.

In other implementations, features of, or used with, the dialysis machine 102 may provide safety features for using the system described here. For example, a pedal 320 in the center of the base of the machine 102 may be implemented to provide a foot brake to prevent motion of the dialysis machine 102. Further, in yet other implementations, frames or guards used in front of the platform for concentrate jugs may serve as a barrier to prevent accidental activations of the sensors 151, 152 when passing by the machine or working in the immediate area. Other guards and safety features to prevent accidental activation of the sensors 151, 152, 153, 154 may also be used. For example, the sensors 151, 152, 153, 154 may only be activated at certain times, and the availability of the sensors 151, 152, 153, 154 to allow an operator to engage with the dialysis machine 102 may be indicated with LED lights on/near them or with an icon on the display 118. Additionally and/or alternatively, the motion sensors 151, 152, 163, 154 may enable different actions based on motion of the operator's hand or foot near different ones of the motion sensors 151, 152, 153, 154. For example, the sensors 151, 152 may correspond to buttons on the touch screen display 118 and the sensors 153, 154 may enable selection or scrolling among different button options.

In another implementation, one or more of the sensors 150, 151, 152, 153, 154 may be configured to recognize a specific gesture in order to activate one or more of the sensors and/or wake-up of the dialysis machine 102 to accept further operator interaction. For example, one more of the sensors 150, 151, 152, 153, 154 may be configured, in accordance with appropriate software, to recognize either a hand wiping motion and/or a foot swiping motion as a required gesture to activate permitted interaction of the operator with the dialysis machine 102 according to the system described herein. This capability may provide for additional safety features of the system described herein by providing for the system described herein to distinguish an intentional activation from a coincidental motion near the dialysis machine 102.

In other implementations, the sensors 151, 152, 153, 154 may also have functions extending beyond muting and resetting alarms. For example, during venous drip chamber level alarms, operator intervention may be required to raise the level in the venous drip chamber. The standard controls to adjust the level may be above the chamber on the touch screen display 118, this requires the operator to hunch down to carefully watch the chamber level through a little window while fumbling with user interface above their head. The motion sensors 151, 152, 153, 154 may include features activated to control the venous drip chamber level. For example, the sensors 153, 154 may enable the operator to wave her hand in gestures that are used to increase or decrease the venous drop chamber level. Other controls actions, such as increasing or decreasing pump speed, may be used and understood for use in connection with the system described herein. Such features would address these usability concerns and reduce the need to change gloves. Additionally, visual and audible cues may be incorporated to facilitate easier verification of the appropriate venous drip chamber level: the illuminated window may change color and a natural- sounding progression of musical chords may sound to indicate the appropriate level is set in order to not contribute to alarm nuisance in a clinic.

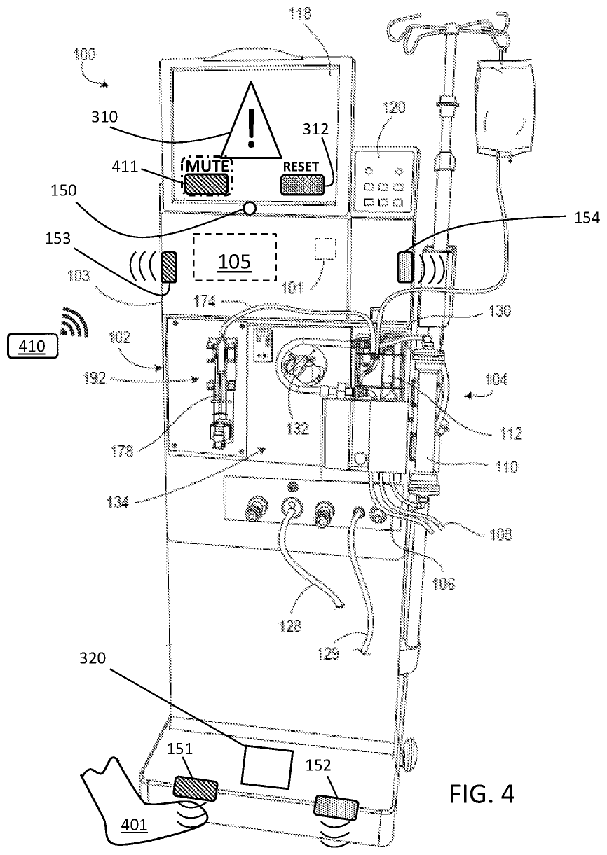

FIG. 4 is a schematic illustration showing an example operation of the system described herein according to an implementation thereof involving contactless interaction with the dialysis machine 102 by an operator 401. As illustrated, the dialysis machine 102 may indicate that an alarm has been triggered, illustrated by display of the visual alarm 310 on the display 118 of the dialysis machine 102. In response to notification of the alarm, the operator 401, such a nurse, patient care technician, or other clinician, may approach the dialysis machine 102. The operator 401 may present credentials 410, such as a smartcard, badge and/or smartphone, that uses short-range wireless technology such as NFC, Bluetooth and/or RFID, to communicate with the proximity sensor 150 to enable identification and authentication of the presented credentials 410 of the operator 401. The operator 401 may then contactlessly interact with the dialysis machine 102 to respond to the alarm 310, such as bringing a hand and/or foot of the operator 401 into proximity of one or more of the sensors 151, 152, 153, 154. As illustrated in the example shown, a foot of the operator 401 is brought into proximity of the sensor 151 that provides the function of muting the alarm, and being the same or similar function of the mute button 411 shown on the touchscreen display 118. As a result, the mute button 411 may be shown highlighted and/or otherwise identified as activated to alert the operator 401 about the action that is being performed at the dialysis machine 102 concerning the alarm 310. Thereafter, the operator 401 may perform an action to respond to the cause of the alarm, including action permitted by different buttons that appear on the display 118, such as a button to raise the venous drip chamber level, and such actions may be performed using gestures in front of the one or more of the motion sensors 151, 152, 154, 154.

In another implementation, the system described may be implemented in the form of a wake-up switch. For example, a dialysis machine 102 may have a “sleep” screen that displays limited information after a delay. To view the full treatment monitoring screen, the operator 401 needs to engage the dialysis machine 102. In accordance with the system described herein, after presenting credentials using the credential device 410, the operator 401 may perform a wake up action of the dialysis machine 102 with a gentle foot motion near one of the motion sensors, such as one of the motion sensors 151, 152 at the base of the dialysis machine 118. This would make performing rounds easier for clinicians—no need to change gloves when checking treatment parameters not shown on the sleep screen. The confirmed identity and contactless wake-up of the dialysis machine may trigger a sub-sleep screen that presents a variety of limited options available that can be performed with further contactless operation, according to the system described herein, without a full wake-up.

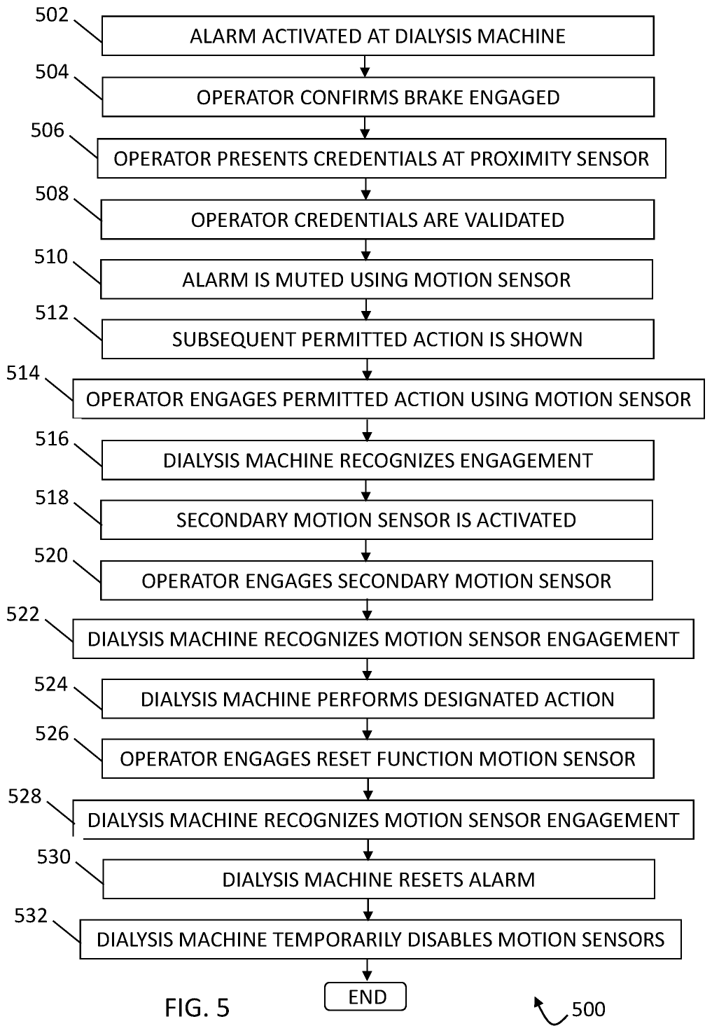

FIG. 5 is a flow diagram 500 showing an example workflow for actions and/or processing performed by, and contactless engagement between, the dialysis machine (e.g. the dialysis machine 102 described herein) and an operator (like the operator 401 described herein) concerning an alarm. At a step 502, an alarm is activated at the dialysis machine, which may be an audible alarm and/or a visual alarm on the display of the dialysis machine. For example, the alarm may indicate that the venous drip chamber level is low; however, other types of alarms are contemplated in connection with the system described herein. At a step 504, a responding operator confirms that a brake of the dialysis machine is engaged. At a step 506, the operator presents credentials in proximity to a proximity sensor, for example by using a credential device with wireless transmission capability, such as by using NFC, Bluetooth and/or RFID. At a step 508, the proximity sensor and/or coupled processor perform identification/credential verification processing. At a step 510, the operator mutes the alarm using one or more motion sensors coupled to the dialysis machine, such as by placing operator's foot near an ultrasonic motion sensor at the base of the dialysis machine, for which the function of the motion sensor matches that of a button shown on a touch screen display of the dialysis machine.

At a step 512, a subsequent permitted action to respond to the alarm is shown on the touchscreen display of the dialysis machine, such as a button to raise the venous drip chamber level, and the corresponding motion sensor at the base of the dialysis machine is designated/illuminated. At a step 514, the operator places her foot or hand against/near the designated motion sensor to activate the permitted action. At a step 516, the dialysis machine recognizes the operator has engaged the button corresponding to the designated motion sensor and performs the action. At a step 518, a secondary motion sensor on the side of the dialysis machine is activated and illuminated. At a step 520, the operator moves her hand in proximity to the secondary motion sensor to engage the sensor. At a step 522, the dialysis machine recognizes the operator has engaged with the secondary motion sensor. At a step 524, the dialysis machine responds to the engagement of the secondary motion sensor to perform an action, such as raising the venous drip chamber level and visually demonstrating it on the display screen with an accompanying audible indication. At a step 526, the operator moves her foot into a motion sensor at the base of the dialysis machine corresponding to resetting the alarm to engage the sensor. At a step 528, the dialysis machine recognizes the engagement of the reset button. At a step 530, the alarm is reset by the dialysis machine. At a step 532, the motion sensors are disabled until the operator's feet move away from the dialysis machine and back to the dialysis machine again to prevent accidental adjustments.

Implementations discussed herein may be combined with each other in appropriate combinations in connection with the system described herein. Additionally, in some instances, the order of steps in the flow diagrams, flowcharts and/or described flow processing may be modified, where appropriate. The system may further include a display and/or other computer components for providing a suitable interface with a user and/or with other computers. Aspects of the system described herein may be implemented or controlled using software, hardware, a combination of software and hardware and/or other computer-implemented or computer-controlled modules or devices having described features and performing described functions. Data exchange and/or signal transmissions to, from and between components of the system may be performed using wired or wireless communication. This communication may include use of one or more transmitter or receiver components that securely exchange information via a network, such as the Internet, and may include use of components of local area networks (LANs) or other smaller scale networks, such as Wi-Fi, Bluetooth or other short range transmission protocols, and/or components of wide area networks (WANs) or other larger scale networks, such as mobile telecommunication networks.

Software implementations of aspects of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media, a memory card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system. The meanings of any method steps of the invention(s) described herein are intended to include any suitable method of causing one or more parties or entities to perform the steps unless a different meaning is expressly provided or otherwise clear from the context.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or similar, should be generally construed to include [a] alone, [b] alone, [c] alone, or any combination of [a], [b] and [c].

mbodiments and implementations of the invention will be apparent to those skilled in the art from a consideration of the specification or practice of the invention disclosed herein. It is intended that the specification and examples be considered as exemplary only, with the true scope of the invention being indicated by the following claims.

1. A medical system for conducting a medical treatment, comprising:
a medical device for performing the medical treatment and having a display; and
at least one motion sensor coupled to the medical device, wherein the at least one motion sensor includes a contactless interface that is configured to enable contactless interaction by an operator with the medical device, wherein the display of the medical device identifies an action at the medical device that is enabled by interaction with the contactless interface of the at least one motion sensor.

2. The medical system according to claim 1, wherein the contactless interface of the at least one motion sensor includes an ultrasonic sensor.

3. The medical system according to claim 1, wherein the at least one motion sensor is disposed at a base of the dialysis machine and configured to respond to motion of a foot of the operator.

4. The medical system according to claim 1, wherein the at least one motion sensor includes at least two motion sensors coupled at different locations on the medical device, and wherein a first motion sensor of the at least two motion sensors corresponds to, and is positioned remotely from, a first button displayed on the display of the medical device, and wherein a second motion sensor of the at least two motion sensors corresponds to, and is positioned remotely from, a second button displayed on the display of the medical device.

5. The medical system according to claim 1, wherein the action includes one or more of muting or resetting an alarm at the medical device.

6. The medical system according to claim 1, wherein the action includes modifying a parameter of the medical device to respond to an alarm at the medical device.

7. The medical system according to claim 1, further comprising a proximity sensor coupled to the medical device, wherein the proximity sensor is configured to receive short-range wireless signals.

8. The medical system according to claim 7, wherein the short-range wireless signals include WiFi, Bluetooth or RFID signals.

9. The medical system according to claim 7, further comprising a credential device that provides a wireless signal containing credential information of the operator to the proximity sensor.

10. The medical system according to claim 9, wherein the credential information of the operator is verified via the medical device before the at least one motion sensor is activated to enable the operator to interact with the contactless interface of the at least one motion sensor.

11. The medical system according to claim 1, wherein the at least one motion sensor is configured to recognize a specific gesture by the operator before enabling further contactless interaction with the by the operator with the medical device.

12. A method for contactless interfacing with a medical device configured to perform a medical treatment, comprising:
displaying an alarm concerning the medical treatment on a display of the medical device;
activating at least one motion sensor that is coupled to the medical device, wherein the at least one motion sensor includes a contactless interface configured to enable contactless interaction by an operator to interact with the medical device to respond to the alarm, wherein an action at the medical device caused by the operator interacting with the contactless interface of the at least one motion sensor is identified on the display of the medical device.

13. The method according to claim 12, wherein the contactless interface of the at least one motion sensor includes an ultrasonic sensor.

14. The method according to claim 12, wherein the at least one motion sensor is disposed at a base of the dialysis machine and configured to respond to motion of a foot of the operator.

15. The method according to claim 12, wherein the at least one motion sensor includes at least two motion sensors coupled at different locations on the medical device, and wherein a first motion sensor of the at least two motion sensors corresponds to, and is positioned remotely from, a first button displayed on the display of the medical device, and wherein a second motion sensor of the at least two motion sensors corresponds to, and is positioned remotely from, a second button displayed on the display of the medical device.

16. The method according to claim 12, wherein the action includes one or more of muting or resetting the alarm at the medical device.

17. The method according to claim 12, wherein the action includes modifying a parameter of the medical device to respond to the alarm at the medical device.

18. The method according to claim 12, further comprising a proximity sensor coupled to the medical device, wherein the proximity sensor is configured to receive short-range wireless signals.

19. The method according to claim 18, wherein the short-range wireless signals include WiFi, Bluetooth or RFID signals.

20. The method according to claim 18, further comprising a credential device that provides a wireless signal containing credential information of the operator to the proximity sensor.

21. The method according to claim 20, wherein the credential information of the operator is verified via the medical device before the at least one motion sensor is activated to enable the operator to interact with the contactless interface of the at least one motion sensor.

22. The method according to claim 12, wherein the at least one motion sensor is configured to recognize a specific gesture by the operator before enabling further contactless interaction with the by the operator with the medical device.
